SEQUENCE LISTING (2 sequences)

SEQ ID NO: 1 - RNA, 21 nt, synthetic construct
  source 1..21: mol_type = other RNA
gtttggcatcaagtacatctt

SEQ ID NO: 2 - RNA, 21 nt, synthetic construct
  source 1..21: mol_type = other RNA
gctaacgcagcagttgcaaac

(19) United States
(12) Patent Application Publication
(10) Pub. No.: US 2026/0315514 A1
(43) Pub. Date: Oct. 15, 2026

(54) Agents to generate repair oligodendrocytes and their use for axonal regrowth

(71) Applicant: Johannes Gutenberg-Universität Mainz, Mainz (DE)

(72) Inventors: Claire Jacob, Mainz (DE); Adrien Vaquié, Botthisham (GB)

(21) Appl. No.: 19/484,039

(22) PCT Filed: May 6, 2024
(86) PCT No.: PCT/EP2024/062461

§ 371 Date: Nov. 13, 2025

(30) Foreign Application Priority Data
May 17, 2023 (EP) 23173982.2

(51) Int. Cl. A61K 31/7105; A61K 31/137; A61K 31/145; A61K 31/225; A61K 31/40; A61K 31/438; A61K 31/506; A61K 31/65; A61P 25/00

(52) U.S. Cl. CPC A61K 31/7105; A61K 31/137; A61K 31/145; A61K 31/225; A61K 31/40; A61K 31/438; A61K 31/506; A61K 31/65; A61P 25/00

(57) ABSTRACT

Therapeutically active agents and methods to activate c-Jun N-terminal kinases (JNK) and/or upregulating/overexpressing the transcription factor c-Jun in oligodendrocytes (OLs) in order to convert mature OLs into repair OLs and thereby promote axonal regrowth after an injury of the central nervous system (CNS). In more particular, the invention relates to activators of JNK and/or c-Jun for the use in the treatment of CNS injuries or CNS diseases that involve axonal disconnection, axonal lesion and/or axonal degeneration. A pharmaceutical composition, comprising an agent that activates JNK and/or c-Jun and has the ability to convert OLs into repair OLs, thereby promoting axonal regrowth after a CNS injury.

### FIELD OF THE INVENTION

The present invention relates to therapeutically active agents and methods with the ability to generate repair oligodendrocytes in order to promote axonal regrowth. More specifically, the invention relates to activators of c-Jun N-terminal kinases (JNK) and/or transcription factor c-Jun which are able to convert mature oligodendrocytes into repair oligodendrocytes and thereby promote axonal regrowth after an injury of the central nervous system (CNS). In more particular, the invention relates to activators of JNK and/or c-Jun for the use in the treatment of CNS injuries or CNS diseases that involve axonal disconnection, axonal lesion and/or axonal degeneration.

### BACKGROUND OF THE INVENTION

Axonal damage is a key predictor of outcome in a number of diverse human diseases of the CNS. Spinal cord injury (SCI) disrupts the structural and functional connectivity between the higher center and the spinal cord, resulting in severe motor, sensory, and autonomic dysfunction with a variety of complications. The pathophysiology of SCI is complicated and multifaceted, and thus individual treatments acting on a specific aspect or process are inadequate to elicit neuronal regeneration and functional recovery after SCI. The pathophysiology of SCI involves primary injury and secondary injury. The primary injury is caused by acute mechanical trauma and results in vascular disruption, blood-spinal cord barrier rupture, cell death (neurons, glial cells, and endothelial cells), and interruption of neural fiber tracts in the spinal cord. The secondary injury references the consecutive pathological events triggered by the primary injury, such as hemorrhage, excitotoxicity, neuroinflammation, demyelination, astrogliosis and extracellular matrix remodeling, which aggravate tissue damage, and compromise neuroplasticity.

Axon regeneration encompasses multiple forms of axon growth, including long-distance axon regrowth, compensatory sprouting of injured and spared supraspinal axons as well as propriospinal neurons, synapse remodeling, and circuit reorganization.

This is in contrast with peripheral axons that can regrow efficiently after injury². These differences are partly due to the different plasticity of myelinating cells, Schwann cells (SCs) and oligodendrocytes (OLs), in these two systems³. The molecular mechanisms underlying this different plasticity remain however poorly understood.

After a SCI, axonal regrowth is highly inefficient and axons fail to regrow, which results in permanent loss of function, thereby greatly altering the quality of life of affected individuals¹.

In contrast, traumatic injuries of the peripheral nervous system (PNS) can be efficiently repaired².

The reasons for these different regenerative properties are multifold:
1. PNS axons have in general a higher intrinsic capacity for regrowth as compared to CNS axons⁷,
2. in the CNS, a glial scar rapidly forms in the lesion site and acts as a barrier for axonal regrowth⁸,
3. the myelin of OLs in the CNS contains several growth-inhibitory factors for axons and thus inhibits axonal regrowth³,
4. axonal fragments in the CNS persist a long time after injury and also act as inhibitors of axonal regrowth⁹⁻¹¹,
5. in contrast to the PNS, there is no guidance mechanism in the CNS allowing axons to reconnect to their former target¹²,
6. remyelination in the CNS, which is mostly achieved by OL precursor cells that need to migrate to the lesion site and be in sufficient number to remyelinate axons¹³, may be more challenging than remyelination in the PNS, which is achieved by SCs that are already in contact with or are present in the vicinity of damaged axons².

In light of the multiple barriers to functional regeneration in the CNS, the combination of several time-controlled regenerative strategies appears to be the most promising approach.

In the spinal cord, axonal disintegration after injury is a lot slower than in the PNS⁹⁻¹⁸. The persistence of axon fragments has been shown to delay axonal regrowth in the PNS and decrease axonal sprouting in the CNS^{20-22,11}. Therefore, accelerating the disintegration of distal cut axons has the potential to facilitate axonal regrowth after injury. In contrast to SCs, OLs in contact with damaged axons fail to demyelinate and their myelin that contains several growth-inhibitory factors for axons, prevents axonal regrowth³. Promoting the removal of myelin debris and inducing demyelination of OLs in contact with damaged axons distal to the lesion site has thus also the potential to enhance axonal regrowth in the CNS.

In summary, regeneration of the CNS after injury is highly inefficient. This is in contrast to the PNS that can efficiently regenerate after injury. These different regenerative properties are in large part due to the different types of glial cells present in these two systems, which create either a pro-regenerative environment in the PNS or in contrast an anti-regenerative environment in the CNS after injury.

### SUMMARY OF INVENTION

It is the object of the present invention to provide therapeutically active agents that are able to generate repair OLs in order to promote axonal regrowth after an injury of the CNS, in particular after a SCI.

This object is solved by activators of JNK and/or transcription factor c-Jun, which are able to trigger conversion of mature OLs into repair OLs.

In a first aspect, the present invention relates to activators of JNK or of c-Jun for the use in the treatment of CNS injuries or CNS diseases that involve axonal disconnection, axonal lesion and/or axonal degeneration.

In a further aspect, the present invention relates to a pharmaceutical composition comprising an agent that activates JNK and/or increases the expression of the transcription factor c-Jun in OLs to convert them into repair OLs and thereby promote axonal regrowth after a CNS injury, and a pharmaceutically acceptable carrier.

In a further aspect, the invention relates to a pharmaceutical composition comprising at least one c-Jun or JNK activator and/or repair OLs, and a pharmaceutical acceptable carrier.

In a further aspect, the invention relates to a pharmaceutical composition for use in the treatment of CNS injuries or CNS diseases that involve axonal disconnection, axonal lesion and/or axonal degeneration.

The invention also relates to an in-vitro method for axonal regrowth after injury, comprising the conversion of OLs into repair OLs by JNK activation and/or c-Jun overexpression in OLs to induce their conversion into repair OLs.

### DESCRIPTION OF INVENTION

The problem underlying the present invention is that, after a CNS lesion, a glial scar forms in the lesion site, the myelin of damaged axons is not removed and the disintegration of damaged axons is very slow.

The c-Jun- or JNK-activating agents of the present invention allow a conversion of OLs into repair cells reminiscent of repair SCs, which actively demyelinate and induce fast disintegration of damaged axons. In addition, repair OLs secrete neurotrophic factors, such as repair SCs. The present invention enables OLs to become repair cells after injury to promote CNS regeneration. The present invention shows that by modifying the activation of one pathway or the expression of one transcription factor, OLs can convert into repair OLs and thereby promote axonal regrowth after a CNS injury.

In a first aspect, the activator of JNK or c-Jun has the ability to convert mature OLs into repair OLs, thereby promoting axonal regrowth after an injury by increasing c-Jun or JNK expression levels or activity.

In a further aspect, the activator of c-Jun has the ability to increase c-Jun expression or c-Jun activity in OLs.

In a further aspect, the activator of JNK has the ability to increase JNK expression or JNK activity in OLs.

Dual specificity phosphatase 6 (Dusp6) is used in the context of the present invention as a target to trigger c-Jun or JNK activation. Dusp6 is a member of the dual specificity protein phosphatase subfamily that inactivates MAP kinases ERK1/2⁴. We found that Dusp6 downregulates the transcription factor c-Jun. After a PNS injury, c-Jun is upregulated in SCs⁵⁻⁶, but it is not upregulated in OLs after a CNS injury. As shown herein, Dusp6 is rapidly downregulated in SCs and upregulated in OLs after injury. Thus, the Dusp6/ERK/c-Jun axis is oppositely regulated in SCs and OLs after injury. Ablation or inactivation of Dusp6 induces rapid ERK1/2 phosphorylation, c-Jun upregulation and filopodia formation in OLs, leading to mechanically-induced, fast disintegration of distal ends of injured axons, myelin clearance and axonal regrowth. Furthermore, Dusp6 has been shown in some instances to dephosphorylate other substrates including c-Jun-N-terminal kinases (JNKs)²³⁻²⁵.

The inventors surprisingly found that an activation of JNK and/or overexpression of c-Jun results in a conversion of OLs into repair OLs. The finding implies that by activation of JNK and/or overexpression of c-Jun, axonal regrowth can be initiated or promoted. An activator of JNK and/or c-Jun is therefore suitable in the treatment of CNS injuries or CNS diseases that involve axonal disconnection, axonal lesion and/or axonal degeneration.

More specifically, it is shown in the present invention that an activation such as an upregulation of c-Jun and/or a phosphorylation of JNK triggers the conversion of OLs into repair OLs, which promotes axonal regrowth. As shown herein, activation of c-Jun and/or JNK can be triggered by Dusp6 downregulation or ERK1/2 activation. More specifically, early after CNS injury, Dusp6 is downregulated in SCs but upregulated in OLs. Preventing Dusp6 expression in OLs leads to c-Jun activation by upregulating c-Jun expression.

In one aspect, the activator of the present invention targets a molecule that results in increased c-Jun expression levels or higher JNK activity. This can either be done by directly activating c-Jun or JNK, or indirectly by inhibiting Dusp6 expression levels in OLs. Activating c-Jun or JNK triggers the conversion of mature OLs into repair OLs and promotes axonal regrowth, especially after SCI.

In a first aspect, the present invention therefore relates to activators of JNK and/or of c-Jun for use in the treatment of CNS injuries or CNS diseases that involve axonal disconnection, axonal lesion and/or axonal degeneration.

Activators of c-Jun or JNK are molecules that stimulate the activity or expression of c-Jun transcription factor or JNK kinases in OLs, thereby enhancing downstream signaling pathways. An activator of JNK or c-Jun thus relates to any molecule, agent, substance or composition that results in increased protein levels of JNK or c-Jun, or increased biological activity of JNK or c-Jun in OLs. As such, an activator of JNK or c-Jun can be an agent that upregulates JNK or c-Jun expression, or an agent that conducts chemical or physical modifications to JNK or c-Jun resulting in higher nucleic acid levels (such as mRNA level), protein levels or c-jun/JNK activity levels. In a preferred embodiment, the agent that activates JNK and/or upregulates the transcription factor c-Jun in OLs to convert OLs into repair OLs is a transcription factor, an enhancer or another modulating agent.

JNK refers to a protein kinase, which induces transactivation of c-Jun. JNK as used in the context of the present invention comprises one or more of the three isoforms of JNK, i.e. JNK1, JNK2, and JNK3, both dephosphorylated and phosphorylated.

In a preferred embodiment, the activator of JNK or of c-Jun is a small molecule or a gene therapy agent.

In a preferred embodiment, the activator of c-Jun is mRNA.

In a preferred embodiment, the mRNA that comprises a nucleotide sequence that is at least partially identical to a nucleotide sequence of c-Jun mRNA, or active parts thereof, is complexed with lipid nanoparticles (LNPs). Active parts of c-Jun refer to truncated forms of c-Jun mRNA that retain the activity of c-Jun as a transcription factor.

In an alternative embodiment, the mRNA is complexed with protein-based nanoparticles or virus-based nanoparticles.

In preferred embodiments the activator activates JNK by phosphorylation at one or more residues, preferably by dual phosphorylation, preferably at T183/Y185 for JNK1 and 2, and T221/Y223 for JNK3.

In preferred embodiments, the activator increases c-Jun expression levels. Preferably, the activator to increase c-Jun expression is an HDAC1/2 inhibitor, including, but not limited to Mocetinostat.

In some embodiments, the activator for c-Jun expression is a regulator of HDAC8 expression, in particular a regulator that mediates HDAC8 downregulation. HDAC8 downregulation increases c-Jun expression. In some embodiments the regulator for HDAC8 downregulation is siRNA.

In some embodiments, the activator of c-Jun or JNK is a Dusp6/Dusp1 inhibitor, preferably BCI (C22H23NO), which controls the biological activity of Dusp6/Dusp1. In alternative embodiments, the c-jun/JNK activator is a Dusp6 inhibitor or Dusp6 downregulator. A preferred Dusp6 inhibitor according to the present invention is shRNA. As shown herein, Dusp6 downregulation by shRNA in purified primary OLs resulted in strongly increased c-Jun levels. Dusp6 downregulation also contributes to SC pro-regenerative behavior. In OLs, this leads to rapid disintegration of distal cut axons and prevents OL-mediated inhibition of axonal regrowth after lesion.

In some embodiments, the activator of c-Jun is a Hif1alpha activator, including, but not limited to prolyl hydroxylase inhibitor dimethyloxalylglycine.

In other embodiments, the activator of JNK is a Hif1alpha activator, including, but not limited to the prolyl hydroxylase inhibitor dimethyloxalylglycine.

In some embodiments, the activator of JNK is Anisomycin, AEBSF hydrochloride, or Azaspiracid-1. Anisomycin is an antibiotic produced by certain Streptomyces bacteria. Anisomycin is a potent activator of JNK, and induces cellular stress responses, leading to the activation of JNK and subsequent phosphorylation of c-Jun.

In alternative embodiments, the activator of c-Jun or JNK relates to phorbol esters, including, but not limited to phorbol 12-myristate 13-acetate (PMA). These compounds activate protein kinase C (PKC), which in turn can activate JNK signaling pathways. PKC-mediated activation of JNK can lead to the phosphorylation and activation of c-Jun.

In alternative embodiments, the activator of c-Jun or JNK relates to cytokines, such as TNF-α, interleukins. Activation of JNK by cytokines can lead to phosphorylation and activation of c-Jun.

The invention also covers truncated or elongated forms of c-Jun or JNK bearing the functional active part thereof. In alternative embodiments, the invention relates to c-Jun or JNK mimetics having similar or identical biological activity as the wild type molecule. Mimetics can be used to enhance c-Jun or JNK activity, or compensate for a lack of c-Jun or JNK expression levels.

In other aspects, the invention also covers compounds that inhibit c-Jun or JNK downregulation or degradation.

The CNS injury or CNS disease treated by the agent or regulator according to the present invention is preferably selected from the group consisting of spinal cord injury, optic nerve injury, traumatic brain injury, stroke, or neurodegenerative diseases of the CNS such as multiple sclerosis, amyotrophic lateral sclerosis, Parkinson's disease, glaucoma. Preferably, the CNS injury is a spinal cord injury.

The present invention also relates to a pharmaceutical composition comprising
a. an agent that activates JNK and/or upregulates the transcription factor c-Jun in OLs to convert OLs into repair OLs and thereby promote axonal regrowth after a CNS injury, or
b. repair OLs, and
c. a pharmaceutically acceptable carrier.

The agents of the invention will positively regulate JNK and/or c-Jun gene expression. In preferred embodiments, the agent is a c-Jun or JNK activator that promotes gene transcription. In some aspects, the agents of the invention are required for gene expression, mRNA transcription, translation or post-translational processes. Preferred activators of the invention are DNA-binding proteins that bind to enhancers or promoter-proximal elements. In preferred embodiments, the activity of the activators of JNK and/or c-Jun can be regulated by a regulator. For example, post-translational modifications to activators can regulate activity, increase or decrease activity depending on the type of modification and activator being modified.

In some embodiments, the pharmaceutical composition according to the present invention comprises
a. an agent that activates JNK and/or upregulates transcription factor c-Jun in OLs to convert OLs into repair OLs and thereby promote axonal regrowth after a CNS injury, or
b. repair OLs, and
c. a pharmaceutically acceptable carrier,
   for use in the treatment of CNS injuries or CNS diseases that involve axonal disconnection, axonal lesion and/or axonal degeneration.

In a preferred embodiment, the pharmaceutical composition comprises an agent that has the ability to activate JNK and/or upregulate transcription factor c-Jun in OLs to convert OLs into repair OLs and thereby promote axonal regrowth after a CNS injury, and a pharmaceutically acceptable carrier.

In an alternative embodiment, the pharmaceutical composition comprises repair OLs, and a pharmaceutically acceptable carrier.

In further preferred embodiments, the pharmaceutical composition according to the present invention comprises an agent that activates JNK and/or upregulates the transcription factor c-Jun in OLs to convert mature OLs into repair OLs and thereby promote axonal regrowth after a CNS injury.

In a preferred embodiment, the agent present in the composition is a pharmaceutically active agent. Preferably, the pharmaceutically active agent in the pharmaceutical composition is an activator of JNK and/or c-Jun as described herein.

In a preferred embodiment, the activator of JNK and/or c-Jun that is present as a pharmaceutically active agent in the pharmaceutical composition is a small molecule or gene therapy agent, including, but not limited to mRNA that is at least partially identical to c-Jun mRNA, or active parts thereof.

In a preferred embodiment, the activator of c-Jun in the pharmaceutical composition is an HDAC1/2 inhibitor, including, but not limited to Mocetinostat.

In a preferred embodiment, the activator of c-Jun or JNK in the pharmaceutical composition is a Hif1alpha activator, including, but not limited to the prolyl hydroxylase inhibitor dimethyloxalylglycine.

In a preferred embodiment, the activator of c-Jun or JNK in the pharmaceutical composition is a regulator that mediates HDAC8 downregulation.

In a preferred embodiment, the activator of JNK in the pharmaceutical composition is selected from the group consisting of Anisomycin, AEBSF hydrochloride, or Azaspiracid-1.

In preferred embodiments, the activator of c-Jun or JNK in the pharmaceutical composition is a Dusp6 inhibitor, including, but not limited to Dusp6-specific shRNA or siRNA.

The pharmaceutical composition of the present invention can include carriers, diluents, excipients or mixtures thereof commonly used in biological preparations. The pharmaceutically acceptable carrier can be any carrier that is able to deliver the composition of the present invention in the living body without limitation such as saline, sterilized water, Ringer's solution, dextrose solution, maltodextrin solution, glycerol, ethanol, or a mixture thereof. If necessary, a general additive such as antioxidant, buffer, and bacteriostatic agent can be added.

When formulating the pharmaceutical composition according to the present invention, generally used diluents or excipients such as fillers, extenders, binders, wetting agents, disintegrating agents and surfactants can be added.

The pharmaceutical composition of the present invention can be formulated as an oral or parenteral preparation. Oral preparations can include solid formulations and liquid formulations. In one aspect, the solid formulation can be tablets, pills, powders, granules, capsules or troches. Such solid formulation can be prepared by adding at least one excipient to the composition. The excipient can be starch, calcium carbonate, sucrose, lactose, gelatine, or a mixture thereof. In addition, the solid preparation can contain lubricants such as magnesium stearate and talc. In preferred embodiments, the liquid formulation can be suspensions, solutions, emulsions or syrups. In this case, the liquid formulation can contain excipients such as wetting agents, sweetening agents, fragrances, and preservatives.

In preferred embodiments, the parenteral preparation can include injections, suppositories, powders for respiratory inhalation, spray aerosols, powders and creams. The injection can include sterilized aqueous solutions, non-aqueous solvents, suspensions, emulsions, and the like. Non-aqueous solvent or suspension, vegetable oils such as propylene glycol, polyethylene glycol and olive oil, or injectable esters such as ethyl oleate can be used. The compositions of the present invention can be administered orally or parenterally according to any desired method. Parenteral administration can include intraperitoneal injection, rectal injection, subcutaneous injection, intravenous injection, intramuscular injection or intrathoracic injection.

The pharmaceutical composition can be administered by the pharmaceutically effective amount to the subject to be treated, i.e. a human or animal. The effective amount can be determined according to the type of CNS disease, the severity, the activity of the drug, the patient's sensitivity to the drug, the time of administration, the route of administration, the duration of treatment, the drugs being used simultaneously, and the like. The composition of the present invention can be administered alone or in combination with other therapeutic agents. In a combination administration, the administration can be sequential or simultaneous.

In alternative embodiments, gene therapy vectors can be used to trigger c-Jun upregulation or JNK activation in OLs. Vectors such as viral vectors are known to introduce genes into a wide variety of different target cells. Typically, the vectors are exposed to the target cells so that transduction can take place in a sufficient proportion of the cells to provide a useful therapeutic or prophylactic effect from the expression of the desired polypeptide. The transduced nucleic acid should not be permanently incorporated into the genome of the targeted cells, to avoid long lasting gene expression. In preferred embodiments, the vector comprises nucleic acid encoding at least the biologically active portion of human or animal c-Jun and/or JNK1, JNK2, JNK3.

As an alternative to the use of viral vectors, other known methods of introducing nucleic acid into cells includes electroporation, calcium phosphate co-precipitation, mechanical techniques such as microinjection, transfer mediated by liposomes and direct DNA uptake and receptor mediated DNA transfer.

The present invention also relates to a pharmaceutical composition according to the present invention for use in the treatment of a CNS injury or CNS disease, including, but not limited to spinal cord injury, optic nerve injury, traumatic brain injury, stroke, or neurodegenerative diseases of the CNS such as multiple sclerosis, amyotrophic lateral sclerosis, Parkinson's disease, glaucoma.

The present invention also relates to an in-vitro method for axonal regrowth after injury, comprising the conversion of OLs into repair OLs by JNK activation and/or c-Jun upregulation in OLs to induce their conversion into repair OLs.

In a preferred embodiment, the in-vitro method comprises a conversion of OLs into repair OLs. Preferably, the conversion is conducted by activation of JNK or upregulation of c-Jun in OLs.

The invention also relates to a method for the generation of repair OLs, comprising the step of conversion of OLs into repair OLs by activating JNK or upregulating c-Jun in said OLs. In a preferred embodiment, the OLs used in the method are generated from human induced pluripotent stem cells. Preferably, the cells are taken from fibroblasts obtained from a skin biopsy of a patient. In preferred embodiments, newly generated OLs are then transduced with adeno-associated viruses (AAVs) expressing c-Jun mRNA or a Dusp6-specific shRNA upon treatment with doxycycline. Activation of JNK or upregulation of c-Jun in said OLs will thus induce the conversion of the OLs into repair OLs. Repair OLs that are generated in this way can then be used for the treatment of CNS injuries or CNS diseases that involve axonal disconnection, axonal lesion and/or axonal degeneration. In a preferred embodiment, repair OLs are a component of a pharmaceutical composition and form part of a cell therapy approach to treat CNS-related disorders. In a preferred embodiment, the composition comprises OLs and doxycycline in addition to the agent that activates JNK and/or upregulates the transcription factor c-Jun in OLs.

In an alternative embodiment, newly generated OLs can be injected in the cerebrospinal fluid of the patient or directly in the glial scar formed after spinal cord injury. In a further step, the patient can be treated with doxycycline to activate the expression of c-Jun or of Dusp6 shRNA in order to produce repair OLs. The thus produced repair OLs will then take action in the treatment of CNS injuries or CNS diseases that involve axonal disconnection, axonal lesion and/or axonal degeneration by promoting CNS regeneration.

In a further preferred embodiment, the method utilizes a regulator that controls the expression of c-Jun or of Dusp6 in cultures of repair OLs treated with doxycycline or its vehicle to confirm efficiency of c-Jun overexpression or of Dusp6 downregulation.

In preferred embodiments, the AAVs carry a resistance gene for an antibiotic and the efficiently transduced cells are selected with said antibiotic.

In summary, the present invention will allow to create a regeneration-compatible environment in the CNS after injury by removing inhibitory cues (damaged myelin and axons) and producing neurotrophic factors. The present invention provides methods and agents that enable the conversion of OLs into repair cells and that will promote CNS regeneration after injury. In preferred embodiments the agent is either an activator of JNK or an mRNA drug that upregulate or overexpress c-Jun or both. More specifically, the invention allows the generation of repair OLs reminiscent of repair Schwann cells by activation of JNK and/or upregulation of c-Jun.

So far, there is no treatment that can convert OLs into repair cells and no treatment that can efficiently promote CNS regeneration after injury. The invention shows that JNK activation and/or c-Jun overexpression in OLs is suitable to induce the conversion into repair OLs and promote CNS regeneration after injury. In some embodiments, JNK can be activated by a small molecule and/or the transcription factor c-Jun can be overexpressed in OLs by an mRNA drug after a spinal cord injury, e.g., during at least the first 5 days after injury. The invention provides the methodology and agents for CNS regeneration after injury, such as, for instance, spinal cord injury. The present invention also comprises embodiments that combine one or more features of specific embodiments described herein. By no means shall the invention be limited to the specific embodiments described in the context of the present invention.

### BRIEF DESCRIPTION OF THE FIGURES

FIG. 1. Dusp6 and phospho-ERK1/2 levels are regulated in Schwann cells (SCs) and OLs after injury.

FIG. 2. Validation of c-jun shRNA and overexpression lentiviruses.

FIG. 3. Dusp6 overexpression in SCs leads to decreased c-Jun expression after axonal lesion.

FIG. 4. The Dusp6/ERK/c-Jun axis is oppositely regulated in SCs and OLs after injury.

FIG. 5. Dusp6 prevents distal cut axon disintegration and axonal regrowth.

FIG. 6. Dusp6 downregulation in OLs induces ERK-dependent distal cut axon disintegration and actin dynamics.

FIG. 7. Dusp6 downregulation in OLs induces c-Jun- and JNK-dependent axonal regrowth.

FIG. 8. Dusp6 ablation in OLs leads to increased phospho-ERK1/2 and c-Jun levels and to fast distal cut axon disintegration after SCI.

FIG. 9. Dusp6 ablation in OLs enables demyelination of damaged axons and axonal regrowth through the glial scar.

### EXAMPLES

The present invention is illustrated in more details in the following examples. By no means shall the invention be restricted to the specific examples. The invention relates to activators of c-Jun or JNK. Dusp6 downregulation and ablation in OLs leads to increased c-Jun expression levels or an activation of the JNK pathway. Increasing the c-Jun expression levels or JNK activity results in axonal regrowth and demyelination of damaged axons.

### Injury-Induced Opposite Regulations in Schwann Cells and Oligodendrocytes

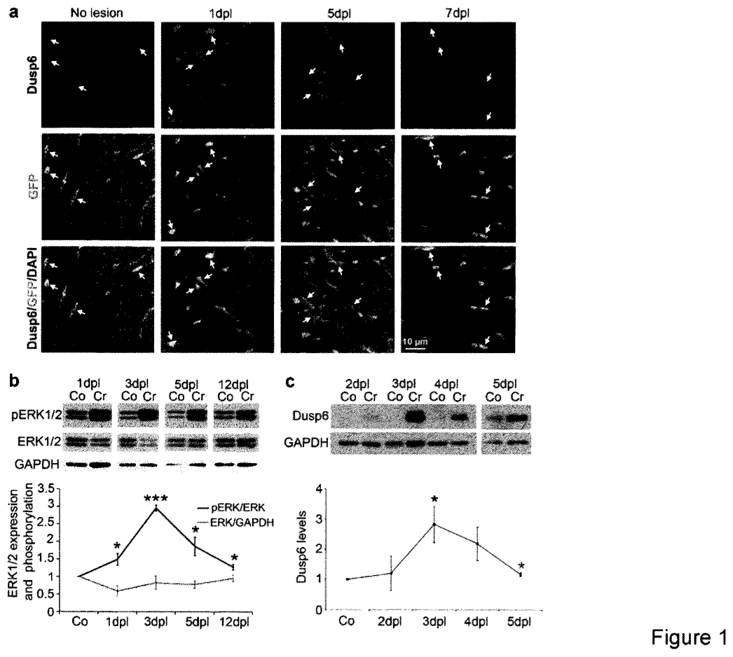

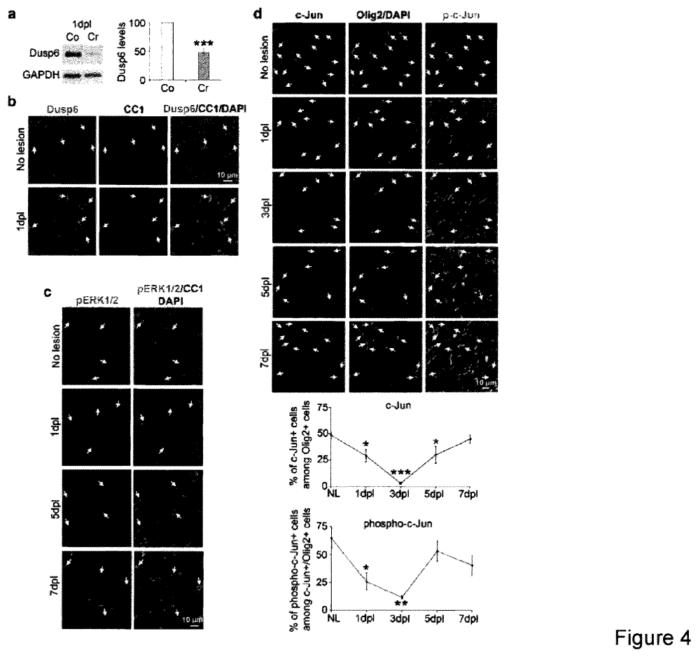

The inventors previously set up and validated microfluidic lesion models of myelinated systems¹⁷. With these models, the inventors analyzed by RNA sequencing mRNA regulations in SCs at 1 day post axonal lesion (dpl) compared to un-lesioned cultures¹⁷. Among the regulated genes revealed by this analysis, the inventors decided to investigate the potential involvement of the phosphatase Dusp6 that was found downregulated in SCs in their microfluidic lesion models¹⁷ and also at 1 day post sciatic nerve crush lesion (SNCL) (FIG. 4a), whereas Dusp6 was upregulated in mature OLs at 1 day post spinal cord hemisection injury (SCI) (FIG. 4b) in mice. Dusp6 upregulation in OLs persisted for 5 days after SCI and levels were decreased at 7 days post SCI (FIG. 1a). ERK1/2 phosphorylation was rapidly increased after SNCL, already at 1 dpl (FIG. 1b), but started only at 7 dpl after SCI (FIG. 4c). ERK1/2 activation is necessary to induce SC demyelination early after peripheral nerve injury but sustained activation of ERK1/2 signaling prevents SC re-differentiation into myelinating SCs and thereby impairs remyelination of regenerated axons¹⁹. It was shown that after a SNCL, ERK1/2 phosphorylation peaks at 3 dpl and then decreases to nearly normalized levels (similar to uninjured nerves) by 12 dpl (FIG. 1b). Consistent with a transient activation of ERK1/2 signaling, Dusp6 levels, after initial downregulation at 1 dpl (FIG. 4a), increased again with a peak at 3 dpl and nearly normalized levels by 5 dpl (FIG. 1c). It was previously shown that the transcription factor c-Jun, which drives SC demyelination together with ERK1/2 signaling and the conversion of mature SCs into repair SCs upon axonal lesion⁶, is rapidly upregulated in SCs after peripheral nerve injury^{26,27}. Simultaneously, JNKs and c-Jun are phosphorylated²⁶. In contrast, it was found that c-Jun was downregulated and the levels of phosphorylated c-Jun decreased in mature OLs after a SCI (FIG. 4d). Taken together, these data show that SCs and OLs react radically differently to injury, with SCs rapidly activating a repair program correlating with Dusp6 downregulation, whereas OLs do not activate this repair program and instead rapidly upregulate Dusp6 expression.

Axon Disintegration and Regrowth in Myelinated Cultures after Lesion

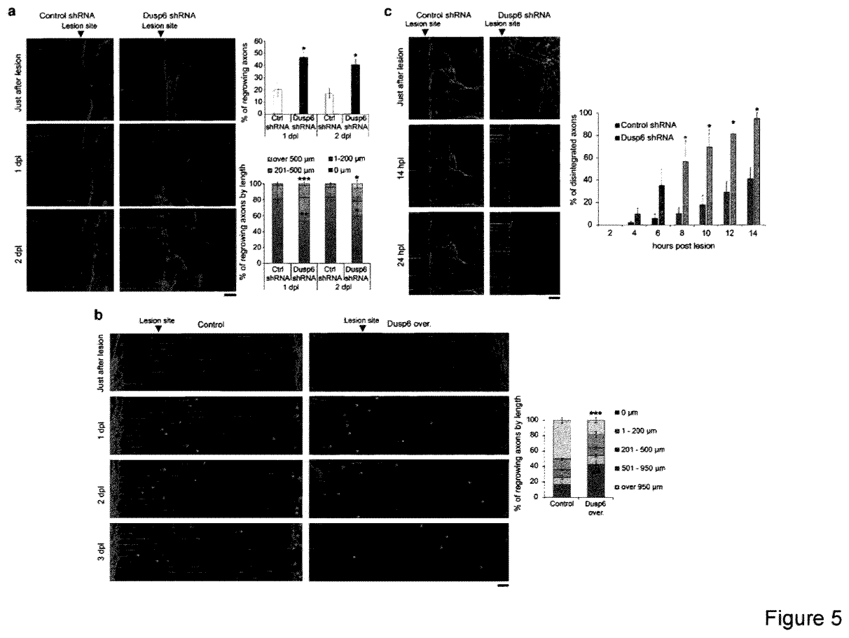

To investigate the function of Dusp6 in OLs after injury, microfluidic lesion models of neuron/OL co-cultures¹⁷ were used. At the myelinated culture stage, Dusp6 was specifically downregulated in OLs by using a lentiviral vector carrying a highly efficient Dusp6-specific shRNA or a non-targeting control shRNA. The inventors previously showed in this microfluidic system that virtually all OLs are efficiently transduced by using highly concentrated lentiviruses in the OL compartment, while neurons do not get transduced or rarely (0-2 neurons per device),^{17,28}. After laser axotomy, the majority of regrowing axons rapidly collapsed and axonal regrowth was overall of low efficiency and over short distance in cultures where OLs received the control shRNA. In comparison, in cultures where OLs received the Dusp6 shRNA, less axons collapsed and axons regrew overall more efficiently, some over long distance (>500 μm) after lesion (FIG. 5a). According to the present invention, Dusp6 is rapidly downregulated in SCs after lesion (FIG. 4a). It was then investigated whether Dusp6 downregulation is involved in SC pro-regenerative behavior after lesion. To answer this question, microfluidic lesion models of neuron/SC co-cultures were used and SCs were transduced with a lentiviral vector expressing Dusp6 to prevent Dusp6 downregulation after laser axotomy. It was found that axonal regrowth was significantly slower in cultures where SCs were transduced with the Dusp6-expressing lentivirus as compared to cultures where SCs received a control lentivirus (FIG. 5b). In addition, the disintegration of distal cut axons was accelerated in cultures where Dusp6 was downregulated by shRNA in OLs compared to cultures where OLs were transduced with a lentivirus carrying a control shRNA (FIG. 5c). These data indicate that Dusp6 downregulation indeed contributes to SC pro-regenerative behavior and in OLs can lead to rapid disintegration of distal cut axons and prevent OL-mediated inhibition of axonal regrowth after lesion.

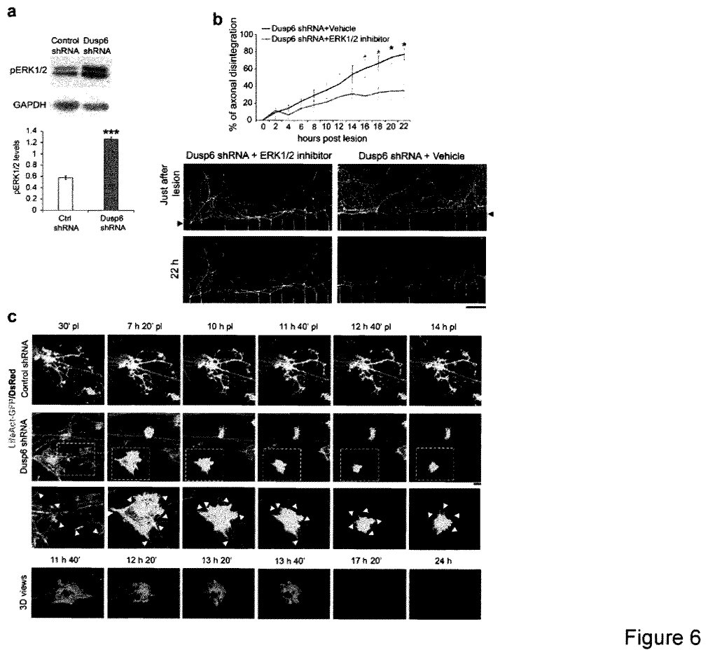

At the molecular level, Dusp6 downregulation by shRNA in purified primary OLs resulted in increased levels of phosphorylated ERK1/2, as expected (FIG. 6a). To test a potential involvement of increased ERK1/2 activation in the rapid disintegration of distal cut axons and the induction of axonal regrowth resulting from Dusp6 downregulation in OLs, the specific ERK1/2 inhibitor MK-8353 were used. It was found that ERK1/2 inhibition in the OL chamber slowed down distal cut axon disintegration and impaired axonal regrowth induced by Dusp6 downregulation in OLs (FIG. 6b). It could thus be concluded that Dusp6 downregulation leads to accelerated distal cut axon disintegration, potentially through increased actin polymerization around distal cut axons. In the microfluidic models used in the present invention, virtually all OLs can be transduced with 2 different high-titer lentiviruses at a 2-day interval between the 2 lentiviruses. OLs were first transduced with lentiviral vectors expressing the Dusp6 shRNA and 2 days later with lentiviral vectors expressing Lifeact-GFP, which dynamically labels F-actin. Interestingly, the shape of OLs where Dusp6 was downregulated by shRNA was altered and actin polymerisation rapidly increased after axotomy, whereas the shape of OLs transduced with the control shRNA lentivirus remained similar and there were no detectable actin polymerisation changes (FIG. 6c). Remarkably, while OLs transduced with the control shRNA lentivirus remained static after lesion with only occasional minor actin process movements and did not form filopodia, OLs transduced with the Dusp6 shRNA lentivirus displayed major actin process changes after lesion, from a flat shape with a few filopodia to a contracted spherical actin shape exhibiting many filopodia (FIG. 6c). Accordingly, time-lapse imaging shows that OLs transduced with the Dusp6 shRNA lentivirus pull distal cut axons until their disintegration (FIG. 6c). After disintegration of distal cut axons, actin filaments in OLs transduced with the Dusp6 shRNA lentivirus depolymerized completely (FIG. 6c).

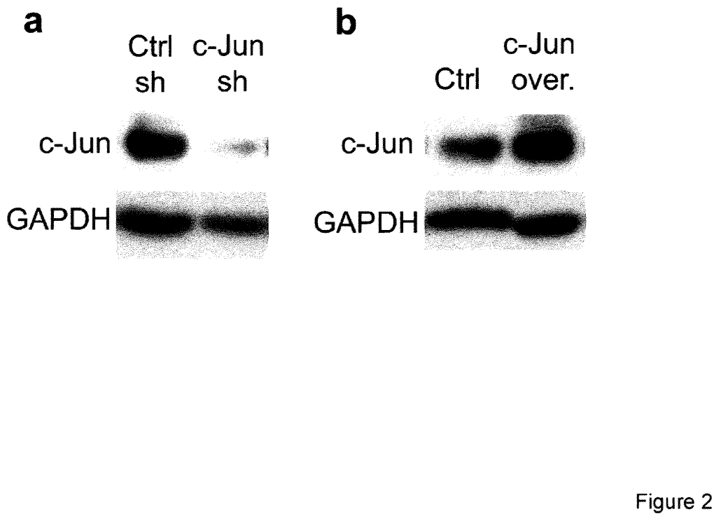

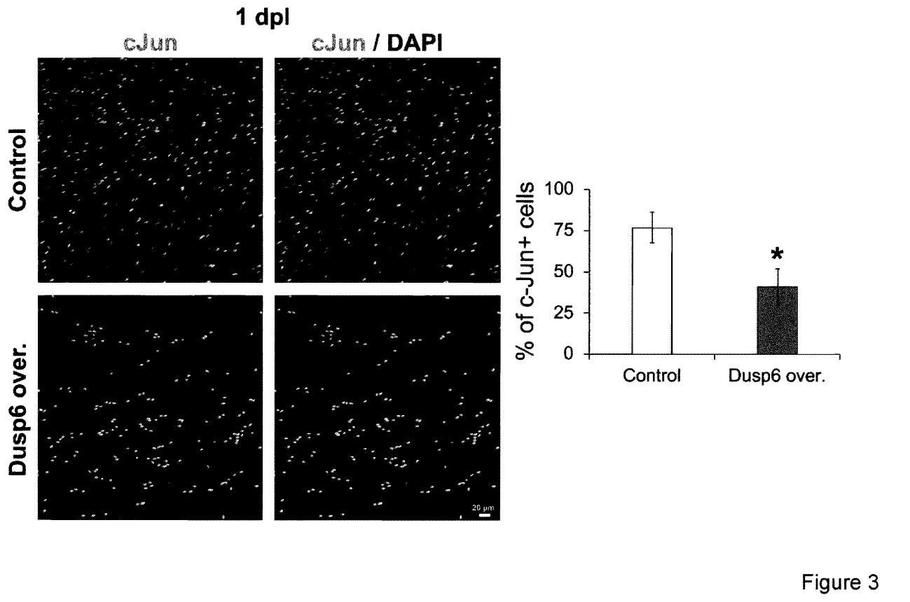

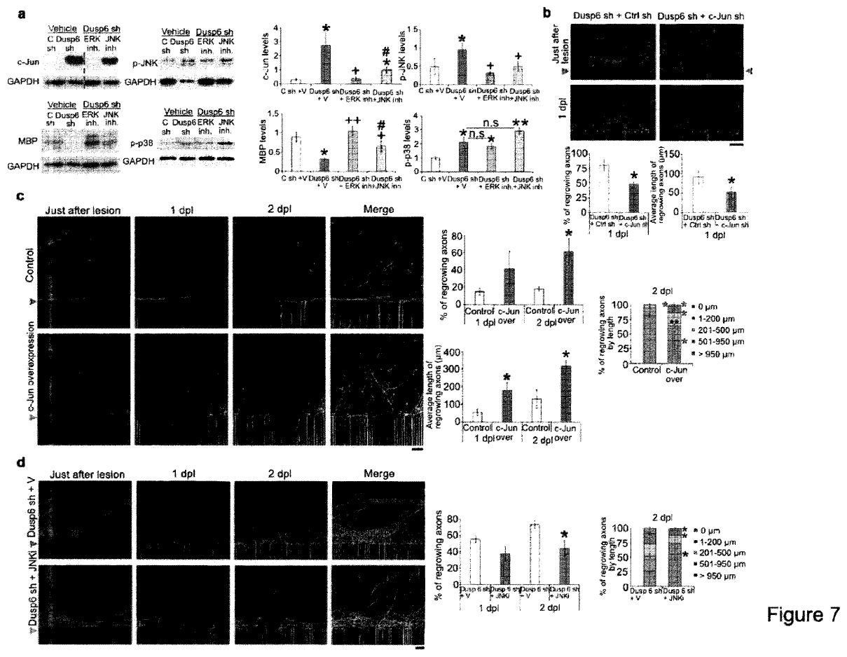

Dusp6 downregulation by shRNA in purified primary OLs resulted in strongly increased c-Jun levels (FIG. 7a). Consistently, Dusp6 overexpression in SCs led to a decreased percentage of c-Jun-positive SCs after axonal lesion (FIG. 3). It was thus investigated whether Dusp6 downregulation in OLs leads to increased c-Jun levels due to increased ERK1/2 activation, and thereby induces a SC-like repair phenotype in OLs after lesion. Indeed, in purified primary OL cultures, c-Jun upregulation induced by Dusp6 downregulation was prevented by ERK1/2 inhibition, while JNK inhibition had a milder but significant effect on c-Jun levels (FIG. 7a). In addition to increasing phospho-ERK1/2 levels, Dusp6 downregulation also increased phospho-JNK levels and this was prevented by ERK1/2 inhibition (FIG. 7a), indicating that the increase of phospho-JNK levels by Dusp6 downregulation is mediated by ERK1/2 activation. Dusp6 downregulation also increased phospho-p38 levels, but this was not prevented by ERK1/2 or JNK inhibition (FIG. 7a). Remarkably, Dusp6 downregulation led to a strong downregulation of MBP expression, which was prevented by ERK1/2 inhibition and to lesser extend by JNK inhibition (FIG. 7a). To test whether Dusp6 downregulation in OLs leads to accelerated distal cut axon disintegration and to the induction of axonal regrowth through increased c-Jun levels, OLs were transduced with lentiviral vectors expressing Dusp6 shRNA and second with lentiviral vectors expressing a c-Jun-specific shRNA or a non-targeting control shRNA. The invention shows that c-Jun downregulation in OLs slows down axonal regrowth but does not affect distal cut axon disintegration induced by Dusp6 downregulation (FIG. 7b). Consistently, c-Jun overexpression in OLs (FIG. 2) did not promote distal cut axon disintegration but induced axonal regrowth after axotomy (FIG. 7c). In addition, JNK inhibition in chambers where Dusp6 was downregulated in oligodendrocytes also affected the percentage of regrowing axons and the percentage of axons regrowing over a long distance (FIG. 7d). Taken together, the invention shows that Dusp6 downregulation allows rapid ERK1/2 activation, leading to c-Jun upregulation and to the acquisition of a SC-like repair phenotype. Interestingly, ERK1/2 activation induced by Dusp6 downregulation promotes axonal regrowth after lesion at least partially via JNK activation and c-Jun upregulation, and induces the disintegration of distal cut axons in a c-Jun-independent manner.

Axon Disintegration and Regrowth after Spinal Cord Injury in Mice

Next, the findings obtained with the microfluidic cell culture models were translated using an in vivo model of SCI. It was investigated whether repair OLs can be induced in vivo and if this is the case, whether these repair OLs can promote distal cut axon disintegration and axonal regrowth after SCI. To address these questions, a tamoxifen-inducible Dusp6 knockout (Dusp6 KO) mouse line was generated where Dusp6 is specifically ablated in the CNS in mature OLs by crossing Dusp6 floxed mice³² with PlpCreERT2 mice³³ expressing the tamoxifen-inducible CreERT2 recombinase under control of the Pip promoter. In some cases, Dusp6 KO mice were crossed with the R26-stop-EYFP reporter mouse line³⁴ to label recombined mature OLs, or with a Thy1-GFP M mouse line³⁵ for sparse labeling of different neuronal subsets. As control mice, PlpCreERT2-negative littermates were used that were treated with tamoxifen at the same time as Dusp6 KO mice.

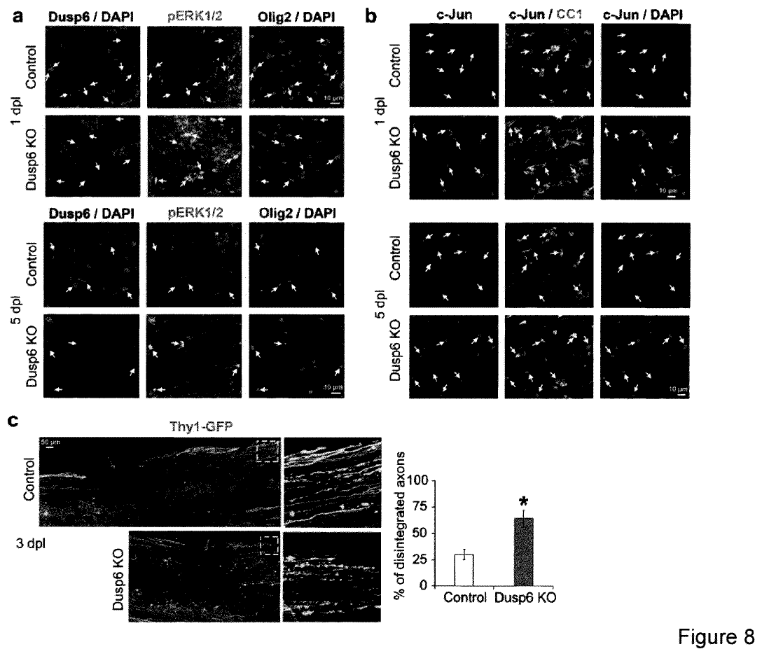

Because Dusp6 expression is below detectable levels in mature OLs of un-lesioned spinal cords, the inventors did not determine the onset of protein loss after tamoxifen, but instead fixed an arbitrary time point of 2 weeks after tamoxifen injection to carry out a SCI in Dusp6 KO and control mice. The invention showed that in wild type mice, Dusp6 is upregulated in mature OLs between 1 and 5 days post SCI (FIG. 1a and FIG. 4b) and that phospho-ERK1/2 is not detected in these cells at these time points (FIG. 4c). In Dusp6 KO mice, Dusp6 was efficiently ablated in OLs with no detectable expression at 1 day or 5 days post SCI (FIG. 8a). Consistently, strong phosphorylated ERK1/2 levels were detected at 1 day post SCI and were sustained until at least 5 days post SCI in mature OLs of Dusp6 KO mice, while ERK1/2 phosphorylation was not detected at these time points in mature OLs of control mice (FIG. 8a). Similarly, c-Jun-expression was also increased in mature OLs at these time points in Dusp6 KO mice compared to control mice (FIG. 8b). Remarkably, the disintegration of distal cut axons below the lesion site was significantly enhanced already at 3 days post SCI in Dusp6 KO mice compared to control mice (FIG. 8c).

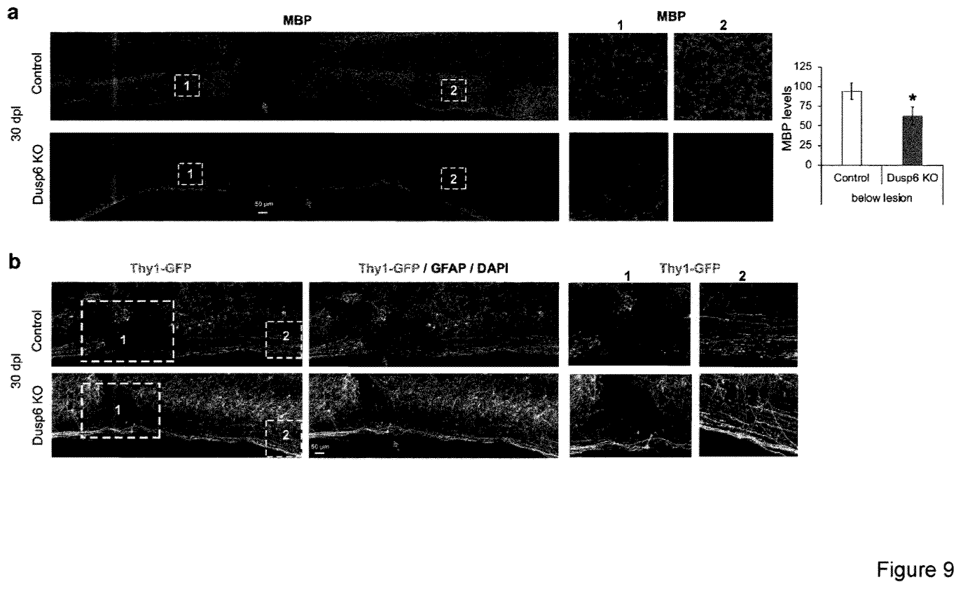

It was then investigated whether myelin is also cleared distal to the SCI site in the absence of Dusp6. Indeed, in Dusp6 KO mice, myelin clearance was increased distal to the lesion site compared to control littermate mice at 1 month post SCI (FIG. 9a). Remarkably, several axons had regrown across the glial scar at the lesion site and distal to the lesion site at 1 month post SCI in Dusp6 KO mice, whereas no axon had grown across the lesion site in control littermates of Dusp6 KO mice (FIG. 9b). Taken together, these data indicate that Dusp6 ablation in mature OLs after SCI allows their conversion into repair OLs, reminiscent of repair SCs after a peripheral nerve lesion. In turn, repair OLs promote distal cut axon disintegration, demyelination distal to the lesion site, and axonal regrowth across and below the lesion site.

The present invention succeeded in converting mature OLs into repair OLs after SCI by mimicking regulations occurring in mature SCs leading to their conversion into repair SCs after a CNS injury.

It was found here that early after lesion, Dusp6 is downregulated in SCs but upregulated in OLs, and that preventing Dusp6 expression in OLs leads to the activation of ERK1/2, JNK and to subsequent upregulation of c-Jun. The invention shows that this is sufficient to induce a SC-like behavior in OLs after lesion. Indeed, ablation of Dusp6 in OLs led to fast disintegration of distal cut axons and enabled axonal regrowth, both in the microfluidic lesion model of neuron/OL cultures and in mice after SCI. In addition, ablation of Dusp6 in OLs resulted in the clearing of myelin debris distal to the SCI site. Thus, inhibitory cues exerted by distal cut axons and OL myelin were removed, creating a favorable CNS environment for axonal regrowth.

In summary, the present invention shows that mature OLs can be converted into repair OLs, reminiscent of repair SCs, upon injury by activation of c-Jun/JNK pathway. Furthermore, the invention shows that repair OLs can promote axonal regrowth after SCI. Finally, the invention provides mechanistic understanding underlying the conversion of mature OLs into repair OLs.

Besides, the present invention also provides a deeper understanding of the mechanisms underlying the different plasticity of SCs and OLs after injury and a method to convert mature OLs exhibiting inhibitory cues for axonal regrowth into repair OLs reminiscent of repair SCs. The invention shows that repair OLs successfully increase the compatibility of the spinal cord environment with axonal regrowth after injury, substantiating the use of repair OLs as therapeutic approach to treat spinal cord injuries. More specifically, the present invention shows that an activation of JNK and/or expression of c-Jun results in a conversion of OLs into repair OLs, which promotes the axonal regrowth. As such, activator of JNK and/or c-Jun are suitable agents in the treatment of CNS diseases that involve axonal disconnection, axonal lesion and/or axonal degeneration or for the treatment of CNS injuries.

### DESCRIPTION OF THE FIGURES

FIG. 1. Dusp6 and phospho-ERK1/2 levels are regulated in Schwann cells and oligodendrocytes after injury. a, Dusp6 and GFP (reporter of recombined mature OLs) co-immunofluorescence and DAPI (nuclei) labeling at 1, 5 and 7 day post spinal cord lesion (dpl) and in un-lesioned (No lesion) mouse spinal cords. Representative images of 3 animals per time point are shown. Arrows point to recombined mature OLs (GFP-positive cells). b,c, Western blots of phospho-ERK1/2 (pERK1/2) and total ERK1/2 (b) or of Dusp6 (c) and quantification normalized to GAPDH at 1-3-5-12 or 2-3-4-5 days post sciatic nerve crush lesion (dpl) in crushed (Cr) compared to contralateral (Co) sciatic nerves of adult mice. Paired one-tailed (grey asterisk) or two-tailed (black asterisks) Student's t-tests, p value: *<0.05, ***<0.001, values=mean, error bars=s.e.m., n=3 animals per time point.

FIG. 2. Validation of c-jun shRNA and overexpression lentiviruses. a,b, Western blots of c-Jun and GAPDH in lysates of primary OLs transduced with lentiviruses expressing either a c-Jun-specific shRNA or a non-targeting control (Ctrl) shRNA (a) or with control empty (Ctrl) lentiviruses or lentiviruses expressing c-Jun (b). Representative images of 3 independent experiments are shown.

FIG. 3. Dusp6 overexpression in Schwann cells leads to decreased c-Jun expression after axonal lesion. C-Jun immunofluorescence and DAPI labeling in chamber #2 (containing Schwann cells) of neuron/SC cultures in microfluidic devices at 1 day post axonal lesion (1 dpl) in chambers where Schwann cells were transduced with lentiviruses expressing Dusp6 or with empty control lentiviruses. The graph shows that percentage of c-Jun-positive cells, n=3 chambers per group, 218 to 450 cells counted per chamber, unpaired two-tailed Student's t-test, p value=*<0.05.

FIG. 4. The Dusp6/ERK/c-Jun axis is oppositely regulated in SCs and OLs after injury. a, Dusp6 Western blot and quantification normalized to GAPDH showing downregulation of Dusp6 at 1 dpl in crushed (Cr) as compared to contralateral (Co) sciatic nerves of adult mice. Paired two-tailed Student's t-tests, p value: ***<0.001, values=mean, error bars=s.e.m., n=3 animals per group. b, Confocal images (z-series projections) of Dusp6 and CC1 (mature OL marker) co-immunofluorescence and DAPI (nuclei) labeling at 1 day post lesion (dpi) and in unlesioned (No lesion) mouse spinal cords. c, Confocal images (z-series projections) of phospho-ERK1/2 (pERK1/2) and CC1 co-immunofluorescence and DAPI labeling at 1, 5 and 7 dpl and in unlesioned mouse spinal cords. d, Confocal images (z-series projections) of c-Jun, phospho-c-Jun (p-c-Jun) and Olig2 (OL marker) co-immunofluorescence and DAPI labeling at 1, 3, 5 and 7 dpl and in unlesioned mouse spinal cords, and percentage of c-Jun-positive cells among Olig2-positive cells and of phospho-c-Jun-positive cells among c-Jun/Olig2-double positive cells. Unpaired one-tailed (grey asterisk) or two-tailed (black asterisks) Student's t-tests, p value: *<0.05, **<0.01,***<0.001, values=mean, error bars=s.e.m., n=3 animals per time point (73 to 181 Olig2-positive cells counted per animal). Arrows show CC1 or Olig2-positive cells (b,c,d).

FIG. 5. Dusp6 prevents distal cut axon disintegration and axonal regrowth. a,c, Time-lapse imaging (wide-field) at different time points after lesion of DsRed-labeled (a) or GFP-labeled (c) axons in microgrooves of neuron/OL cultures where OLs were transduced with lentiviruses carrying either a Dusp6-specific shRNA or a non-targeting control shRNA. Note that lesions were carried out at the exit of microgrooves to quantify axonal regrowth (a) and the percentage of disintegrated distal cut axons (c) in the chamber where OLs interact with axons. In (a), the upper graph shows the percentage of regrowing axons and the lower graph shows the length distribution of axonal regrowth after lesion. b, Time-lapse imaging (wide-field) at different time points after lesion of DsRed-labeled axons in microgrooves of neuron/SC cultures where SCs were transduced with lentiviruses expressing Dusp6 (Dusp6 over.) or with control lentiviruses (empty vector), and graph showing the length distribution of axonal regrowth after lesion. Blue asterisks indicate the position of the tip of axons in the microgrooves and at their exit of the microgrooves. (a,b,c) Fifty-two to 68 axons (a), 76 to 192 axons (b), and 27 to 81 axons (c) were quantified per chamber per day, n=4-6 chambers per group, unpaired one-tailed (grey asterisk) or two-tailed (black asterisks) Student's t-test, p value: *<0.05, **<0.01, ***<0.001, values=mean, error bars=s.e.m. Scale bar, 50 μm (a,b), 100 μm (c).

FIG. 6. Dusp6 downregulation in OLs induces ERK-dependent distal cut axon disintegration and actin dynamics. a, phospho-ERK1/2 (pERK1/2) Western blot and quantification normalized to GAPDH showing increased pERK1/2 levels in primary differentiated OLs transduced with a Dusp6-specific shRNA lentivirus compared to a non-targeting control shRNA lentivirus. Unpaired two-tailed Student's t-tests, p value: ***<0.001, values=mean, error bars=s.e.m., n=3 independent experiments. b, Time-lapse imaging (wide-field) at different time points after lesion of DsRed-labeled axons in microgrooves and chamber #2 of neuron/OL cultures where OLs were transduced with lentiviruses carrying a Dusp6-specific shRNA and treated either with the specific ERK1/2 inhibitor MK-8353 or its vehicle, and quantification of distal cut axon disintegration every two hours for 22 hours. The integrated density (mean grey value) of labeled axons in the 3 same ROI per chamber was averaged at each time point, n=3 chambers per group, unpaired one-tailed (grey asterisks) or two-tailed (black asterisks) Student's t-test, p value: *<0.05, values=mean, error bars=s.e.m. Scale bar, 100 μm. c, Time-lapse confocal imaging at different time points after lesion of DsRed-labeled axons and LifeAct-GFP-labeled OLs (+NucBlue™ Live ReadyProbes™ Reagent shown in magnifications of Dusp6 shRNA-transduced OLs to label nuclei) in chamber #2 of neuron/OL cultures where OLs were transduced with lentiviruses carrying either a Dusp6-specific shRNA or a non-targeting control shRNA. The two upper rows are z-series projections, the third row is a magnification of the region highlighted by a dashed white box in the second row, and the 4^{th} row shows 3D views at different time points starting from 11 h 40′ after lesion of the region highlighted by a dashed white box. The white arrowheads point to filopodia structures. Scale bar=10 μm.

FIG. 7. Dusp6 downregulation in OLs induces c-Jun- and JNK-dependent axonal regrowth. a, c-Jun, phospho-JNK (p-JNK), phospho-p38 (p-p38) and MBP Western blots and quantification normalized to GAPDH in primary differentiated OLs transduced with a Dusp6-specific shRNA lentivirus (Dusp6 sh) or a non-targeting control shRNA lentivirus (C sh) and treated with the specific ERK1/2 inhibitor MK-8353 (ERK inh) or the specific JNK1/2 inhibitor JNK-IN-8 (JNK inh) or their vehicle (V). Unpaired or paired (pJNK: Dusp6 sh/C sh and Dusp6 sh+JNK inh/Dusp6 sh+V; MBP: Dusp6 sh+JNK inh/Dusp6 sh+ERK inh) one-tailed (grey asterisks, crosses or hashtags) or two-tailed (black asterisks or crosses) Student's t-tests, p values: *,+, #<0.05, **,++<0.01; asterisks indicate significance compared to C sh+V, crosses indicate significance compared to Dusp6 sh+V, hashtags indicate significance compared to Dusp6 sh+ERK inh; n.s.=non-significant, values=mean, error bars=s.e.m., n=3 independent experiments. b, Wide-field imaging just after lesion and at 1 dpi of DsRed-labeled axons in microgrooves and chamber of neuron/OL cultures where OLs were transduced with lentiviruses carrying a Dusp6-specific shRNA and either a non-targeting control shRNA or a c-Jun-specific shRNA, and graphs showing the percentage and the average length of regrowing axons. c,d, Wide-field imaging just after lesion (red), at 1 dpl (green) and 2 dpl (turquoise) and merge images of all time points of DsRed-labeled axons in microgrooves and chamber of neuron/OL cultures where OLs were transduced with (c) lentiviruses carrying a c-Jun overexpressing lentivirus or a control (empty backbone) lentivirus or (d) a Dusp6 shRNA lentivirus and treated with a JNK inhibitor (JNKi) or its vehicle, and graphs showing the percentage and average length of all regrowing axons at 1 dpi and 2 dpl and the percentage of regrowing axons by length category at 2 dpl. In (b,c,d), lesions were carried out near the exit of the microgrooves (blue arrowheads). Blue asterisks in (b) indicate regrowing axons. Thirty-eight to one-hundred and eighty axons were quantified per chamber per time point, n=3 chambers per group, unpaired (% of regrowing axons) one-tailed (grey asterisks) or two-tailed (black asterisks) Student's t-test, p values: *<0.05, **<0.01, values=mean, error bars=s.e.m. Scale bar, 50 μm (b), 100 μm (c,d).

FIG. 8. Dusp6 ablation in OLs leads to increased phospho-ERK1/2 and c-Jun levels and to fast distal cut axon disintegration after SC. a, b, Confocal images (z-series projections) of phospho-ERK1/2 (pERK1/2), Dusp6 and Olig2 (OL marker) co-immunofluorescence (a) or c-Jun and CC1 (mature OL marker) co-immunofluorescence (b) and DAPI labeling at 1 and 5 dpl in spinal cords of Dusp6 KO and control mice. White arrows point to OLs. c, confocal imaging and z-series projections of Thy1-GFP-labeled neurons at 3 dpi in Dusp6 KO and control mice and quantification of axonal disintegration below the lesion (17-72 counted axons per ROI, 3 ROI per animal quantified below lesion site, n=3 animals per group), unpaired two-tailed Student's t-test, p value: *<0.05. Orange arrows indicate the lesion site. The spinal cord regions on the right side of the orange arrows are below the lesion site. Images on the right side are magnifications of the regions highlighted by a dashed white box on the left images.

FIG. 9. Dusp6 ablation in OLs enables demyelination of damaged axons and axonal regrowth through the glial scar. a, Confocal images (z-series projections) of MBP immunofluorescence on Dusp6 KO and control mouse spinal cords at 30 days post SCI (30 dpi) and graph showing the average intensity of MBP signal below the lesion site in the white matter (3 ROI averaged per animal). b, Confocal images (z-series projections) of Thy1-GFP-labeled neurons and GFAP (astrocyte/glial scar marker) immunofluorescence and DAPI labeling (nuclei) on Dusp6 KO and control mouse spinal cords at 30 dpl. The GFAP staining delineate the glial scar. Representative images are shown, n=3 animals per group, paired one-tailed Student's t-test, p value: *<0.05. Images on the right side labeled 1 and 2 are magnifications of the regions highlighted by dashed white boxes on the left images. Orange arrows point to the lesion site. The spinal cord regions on the right side of the orange arrows are below the lesion site.

### Material and Methods

### Statistical Analyses

For each data set presented, experiments were performed at least 3 times and p values were calculated using two-tailed (black asterisks, crosses or hashtags) or one-tailed (grey asterisks, crosses or hashtags) Student's t-tests. P values: *<0.05, **<0.01, ***<0.001, values=mean, error bars=s.e.m. Sample size was determined by the minimal number of animals or individual experiment required to obtain statistically significant results and increased in some cases to improve confidence in the results obtained. No animal or data point was excluded from the analysis.

### Animals

To induce ablation of Dusp6 in mature OLs of adult mice, Dusp6 floxed mice³² were crossed with mice expressing a tamoxifen-inducible Cre recombinase under control of the OL-specific Pip promoter³³ (PlpCreERT2). To ablate Dusp6, mice received daily injections of 2 mg tamoxifen (Sigma) for five consecutive days. In some cases, these mice were additionally crossed with the R26-stop-EYFP reporter mouse line³⁴ to label recombined mature OLs or with a Thy1-GFP M mouse line³⁵ to label a fraction of different neuronal subsets. In other cases, the Thy1-GFP M reporter lines was used alone (without other transgene) and in other cases, the reporter line R26-stop-EYFP was crossed to the PlpCreERT2 mouse line only. Genotypes were determined by PCR on genomic DNA.

This study complies with all relevant ethical regulations concerning animal use, which was approved by the Veterinary office of the Canton of Fribourg, Switzerland and the Veterinary office (Landesuntersuchungsamt) of Rheinland-Pfalz, Germany.

### Surgical Procedures

For all surgical procedures, the inventors used isoflurane (3% for induction, 1.5-2% for narcosis during the operation) for anesthesia. For analgesia, 0.1 mg/kg/body weight buprenorphine (Temgesic; Essex Chemie) was administered by i.p. injection 1 h before surgery and every 4 hours (minimum 2 injections and maximum 3 injections) the day of surgery. The day after surgery 1 ml agarose gel containing 0.027 mg/ml buprenorphine was fed twice a day (morning and evening). Sciatic nerve crush lesions were carried out on 3 to 4-months old adult mice (males and females), as previously described²⁷. Spinal cord hemisections were carried out at T8 level on 3 to 4-months old adult mice (males and females) as described⁴⁴. To ensure a complete hemisection, a 36 G needle was inserted at the midline of the spinal cord and moved carefully towards the left until the edge of the spinal cord. No randomization method was used, but experimenters were blinded to the experimental group and received only the animal number given at birth by the animal caretaker.

### Microfluidic Devices

The microfluidic lesion models of neuron/SC and neuron/OL cultures were previously described by the inventors.^{17,28}.

### DRG Neuron/SC Myelinated Cultures

Dorsal root ganglion (DRG) explant was isolated from embryonic day 14.5 (E14.5) Wistar rat embryos, dissociated and cultured as previously described^{17,45}.

### DRG Neuron/OL Myelinated Cultures

DRG explant from E14.5 rat embryos were isolated and neurons were dissociated and cultured in microfluidic devices as previously described⁷.

### Live Imaging, Laser Axotomy and Image Processing

For live-imaging, neurons were labeled either in red or green by adding to chamber #1 (chamber containing neuronal cell bodies) 0.5-2 μL of highly concentrated lentivirus generated as previously described¹⁷. Red fluorescence was obtained by transducing neurons with lentiviruses expressing DsRed under control of the neuron-specific Synapsin promoter. Green fluorescence was obtained by transducing neurons with lentiviruses expressing GFP under control of the CMV promoter. OLs F-Actin structure was labelled in green by adding to chamber #2 0.5-2 μL of highly concentrated Lifeact-GFP lentivirus under control of the CMV promoter. Twenty-four hours before carrying out the laser axotomy all medium was replaced with Minimum Essential Medium (MEM). All axons of the device passing through the microgrooves were lesioned with a laser ablation module as described⁷. Laser axotomy was conducted with a 40× or a 60× Water NA1.25 Apochromat objective.

For 2D and 3D time-lapse imaging, the inventors used a VisiScope spinning disk confocal microscope CSU-W1 (Visitron) to acquire wide-field or confocal images at 20, 30 or 60 min intervals on several stage positions (5 to 10 with at least 10% image frame overlap) during 24 to 48 h with a 40× Oil NA1.25 Apochromat objective or at 24 h intervals on 12 to 60 stage positions during 2 to 4 days with a 20× Air NA0.75 PlanApochromat objective. For 3D time-lapse imaging, optical sections of 0.3 to 0.6 μm thickness (between 30 and 100 stacks) were acquired. For confocal imaging, optical sections of 0.6 to 1.5 μm thickness (between 25 and 100 stacks) were acquired. Multiple stage positions were automatically stitched by processing with the Visiview software (Visitron) and different channels were merge with Fiji and/or Adobe Photoshop (CC 20.0.8 Release). Specific Macro were written to convert the saved.stk or .ome.tf2 raw data to RGB mode, create hyperstacks and adjust brightness and contrast using defined minimal and maximal values and convert to .tif files.

### Primary Rat Oligodendrocyte Cultures

Rat primary oligodendrocytes precursors cells (OPCs) were isolated from neonatal rat neocortices and cultured as described⁴⁶. OPCs were first differentiated for 12 days, then the Dusp6 shRNA or non-targeting control shRNA lentiviruses were added and cells were kept in the differentiation medium for 5 additional days before analysis. In some cases, cells were treated for 2 more days in differentiation medium with 1 μM JNK1/2 inhibitor (JNK-IN-8, MCE, #1410880-22-6), 300 nM ERK1/2 inhibitor (MK-8353, MCE, #HY-111407), and then analyzed.

### Primary Rat SC Cultures

Primary rat SC cultures derived from P2 Wistar rat sciatic nerves were purified, dissociated and cultured as previously described^{27,47}.

### Generation of Lentivirus

Highly concentrated lentiviral particles were produced as previously described (Brugger et al., 2015, Vaquié et al., 2019). Constructs used to produce lentiviruses: packaging constructs pLP1, pLP2 and pLP/VSVG (Invitrogen), pLV-LSyn-RFP⁴⁸ (Addgene construct #22909), pLentiLox 3.7 (ATCC), Lifeact-GFP (kind gift from Dr. Olivier Pertz, University of Bern, Switzerland), Dusp6³⁶ (Addgene construct #27975), Dusp6 shRNA (Sigma, mission shRNA, TRCN0000317759: GTTTGGCATCAAGTACATCTT), c-Jun shRNA (Sigma, mission shRNA, TRCN0000229527: GCTAACGCAGCAGTTGCAAAC) or a non-targeting control shRNA (Sigma, SHC001, MISSION pLKO.1-puro Empty Vector Control), c-Jun (Origene, #RC209804L4) or empty backbone control.

### Immunofluorescence

For immunofluorescence in microfluidic chambers, cells were fixed 30 min with 4% paraformaldehyde (PFA, Sigma) at room temperature (RT), and washed 3 times 15 min with PBS. Cells were subsequently blocked for 3 hrs with blocking buffer (0.3% Triton X-100, 5% BSA, PBS), and then incubated for 2 days at 4° C. with primary antibodies in blocking buffer. Cells were then washed three times 30 min with blocking buffer and incubated for 6 hrs in the dark with secondary antibodies in blocking buffer. After three washes of 30 min with blocking buffer, cells were incubated with DAPI for 30 min. Finally, cells were washed for 30 min and stored in PBS. Imaging was performed briefly after the end of the immunostaining.

For immunofluorescence on spinal cords, mice were deeply anesthetized with a lethal dose of pentobarbital and perfused with 4% PFA after blood removal with heparin. Spinal cords were collected 2 mm above and below the lesion site, post-fixed in 4% PFA for 3 h at RT, incubated in 20% sucrose overnight at 4° C., embedded in O.C.T. compound, and frozen at −80° C. The inventors used 20 to 200 μm-thick cryosections. Twenty to fifty μm-thick cryosections were first submitted to antigen retrieval in citrate buffer (10 mM citrate buffer, 0.05% Tween 20, pH 6.0) for 2 h at 65° C., washed, blocked in blocking buffer (0.1% Triton X-100, 5% BSA, PBS) for 60 min at RT and incubated overnight at 4° C. with primary antibodies diluted in blocking buffer. Sections were then washed 3 times in blocking buffer and secondary antibodies were incubated for 1 h at RT in the dark. Sections were then washed, incubated with DAPI for 10 min at RT, washed again and mounted in Citifluor (Agar Scientific). One hundred to two hundred-μm thick cryosections were permeabilized for 3 h in blocking buffer (1% Triton X-100, 10% FBS, PBS), and then incubated for 2 days at 4° C. with primary antibodies diluted in blocking buffer. After 3 washes of 15 min with blocking buffer, sections were incubated with secondary antibodies overnight at 4° C. in the dark. Sections were then washed 3 times for 15 min with blocking buffer, incubated with DAPI for 1 min and incubated overnight in 70% Glycerol/PBS for clearing. Sections were finally washed with PBS and mounted in CitiFluor.

Primary antibodies: Olig2 (Goat, 1:200, R&D Systems, AF2418), MBP (rat, 1:50. Serotec, cat. #MCA409S), CC1/APC (mouse, 1:200, Millipore, cat. #OP80), DUSP6/MKP3 (Rabbit, 1:200, Invitrogen, ARC0237, #MA5-35048), DUSP6/MKP3 (Mouse, 1:200, Santa Cruz, F-12, #sc-377070), DUSP6 (Rabbit, 1:200, Abcam, ab76310), c-Jun (rabbit, 1:200, Abcam, cat. #ab32137), c-Jun (Rabbit, 1:200, Cell signaling, 60A8, #9165), c-Jun (mouse, 1:200, BD Bioscience, cat. #610327), Phospho-c-Jun (Rabbit, 1:100, Cell Signaling, D47 G9, #3270), Phospho-p44/42 MAPK (Rabbit, 1:200, Cell signaling, D13.1.4E, #4370).

### Western Blot Analysis

Mouse sciatic nerves and primary rat OLs and primary rat SCs were lysed and processed for Western blot analysis as previously described⁴³. Primary antibodies: DUSP6 (Rabbit, 1:1000, Abcam, ab76310), DUSP6/MKP3 (Rabbit, 1:350, Invitrogen, ARC0237, #MA5-35048), DUSP6/MKP3 (Mouse, 1:500, Santa Cruz, F-12, #sc-377070), c-Jun (Rabbit, 1:500, Cell signaling, 60A8, #9165), Phospho-c-Jun (Rabbit, 1:500, Cell Signaling, D47 G9, #3270), p44/42 MAPK (Mouse, 1:1000, Cell signaling, L34F12, #4696), Phospho-p44/42 MAPK (Rabbit, 1:500, Cell signaling, D13.1.4E, #4370), Phospho-JNK1/2 (Rabbit, 1:500, Invitrogen, #44-682 G), MBP (rat, 1:750. Serotec, cat. #MCA409S), GAPDH (glyceraldehyde-3-phosphate-dehydrogenase, mouse, 1:5000, Genetex, cat. #GTX28245, lot #821705388), Phospho-p38 (Rabbit, 1:500, Proteintech, #28796-1-AP).

### REFERENCES

1. Zheng, B. & Tuszynski, M. H. Regulation of axonal regeneration after mammalian spinal cord injury. Nat. Rev. Mol. Cell. Biol. doi: 10.1038/s41580-022-00562-y (2023). Online ahead of print
2. Nocera, G. & Jacob, C. Mechanisms of Schwann cell plasticity involved in peripheral nerve repair after injury. Cell. Mol. Life Sci. 77, 3977-3989 (2020).
3. Brosius Lutz, A. & Barres, B. A. Contrasting the glial response to axon injury in the central and peripheral nervous systems. Dev. Cell. 28, 7-17 (2014).
4. Caunt, C. J. & Keyse, S. M. Dual-specificity MAP kinase phosphatases (MKPs): shaping the outcome of MAP kinase signalling. FEBS J. 280, 489-504 (2013).
5. Harrisingh, M. C. et al. The Ras/Raf/ERK signalling pathway drives Schwann cell dedifferentiation. EMBO J. 23, 3061-3071 (2004).
6. Arthur-Farraj, P. J. et al. c-Jun reprograms Schwann cells of injured nerves to generate a repair cell essential for regeneration. Neuron 75, 633-647 (2012).
7. Huebner, E. A. & Strittmatter, S. M. Axon regeneration in the peripheral and central nervous systems. Results Probl. Cell Differ. 48, 339-351 (2009).
8. Bradbury, E. J. & Burnside, E. R. Moving beyond the glial scar for spinal cord repair. Nat. Commun. 10, 3879 (2019).
9. Buss, A. et al. Sequential loss of myelin proteins during Wallerian degeneration in the human spinal cord. Brain 128, 356-364 (2005).
10. Williams, P. R. et al. A recoverable state of axon injury persists for hours after spinal cord contusion in vivo. Nat. Commun. 5, 5683 (2014).
11. Collyer, E. et al. Sprouting of axonal collaterals after spinal cord injury is prevented by delayed axonal degeneration. Exp. Neurol. 261, 451-461 (2014).
12. Dun, X. P. & Parkinson, D. B. Classic axon guidance molecules control correct nerve bridge tissue formation and precise axon regeneration. Neural Regen. Res. 15, 6-9 (2020).
13. Tepavčević, V. & Lubetzki, C. Oligodendrocyte progenitor cell recruitment and remyelination in multiple sclerosis: the more, the merrier? Brain 145, 4178-4192 (2022).
14. Beirowski, B. et al. The progressive nature of Wallerian degeneration in wild-type and slow Wallerian degeneration (WldS) nerves. BMC Neurosci. 6, 6 (2005).
15. Vargas, M. E. & Barres, B. A. Why is Wallerian degeneration in the CNS so slow? Annu. Rev. Neurosci. 30, 153-179 (2007).
16. Wong, K. M., Babetto, E. & Beirowski, B. Axon degeneration: make the Schwann cell great again. Neural Regen. Res. 12, 518-524 (2017).
17. Vaquié, A. et al. Injured axons instruct Schwann cells to build constricting actin spheres to accelerate axonal disintegration. Cell Rep. 27, 3152-3166 (2019).
18. Gomez-Sanchez, J. A. et al. Schwann cell autophagy, myelinophagy, initiates myelin clearance from injured nerves. J. Cell Biol. 210, 153-68 (2015).
19. Napoli, I. et al. A central role for the ERK-signaling pathway in controlling Schwann cell plasticity and peripheral nerve regeneration in vivo. Neuron 73, 729-742 (2012).
20. Brown, M. C., Lunn, E. R. & Perry, V. H. Consequences of slow Wallerian degeneration for regenerating motor and sensory axons. J. Neurobiol. 23, 521-536 (1992).
21. Chen, S. & Bisby, M. A. Impaired motor axon regeneration in the C57BL/Ola mouse. J. Comp. Neurol. 333, 449-454 (1993).
22. Martin, S. M., O'Brien, G. S., Portera-Cailliau, C. & Sagasti, A. Wallerian degeneration of zebrafish trigeminal axons in the skin is required for regeneration and developmental pruning. Development 137, 3985-3994 (2010).
23. Ramkissoon, A. et al. Targeted inhibition of the dual specificity phosphatases DUSP1 and DUSP6 suppress MPNST growth via JNK. Clin. Cancer Res. 25, 4117-4127 (2019).
24. Relav, L., Estienne, A. & Price, C. A. Dual-specificity phosphatase 6 (DUSP6) mRNA and protein abundance is regulated by fibroblast growth factor 2 in sheep granulosa cells and inhibits c-Jun N-terminal kinase (MAPK8) phosphorylation. Mol. Cell Endocrinol. 531, 111297 (2021).
25. Wu, L. et al. LncRNATCONS_00145741 Knockdown Prevents Thrombin-Induced M1 Differentiation of Microglia in Intracerebral Hemorrhage by Enhancing the Interaction Between DUSP6 and JNK. Front. Cell Dev. Biol. 9, 684842 (2022).
26. Parkinson, D. B. et al. c-Jun is a negative regulator of myelination. J. Cell Biol. 181, 625-637 (2008).
27. Brugger, V. et al. Delaying histone deacetylase response to injury accelerates conversion into repair Schwann cells and nerve regeneration. Nat. Commun. 8, 14272 (2017).
28. Vaquié, A., Sauvain, A. & Jacob, C. Modeling PNS and CNS Myelination Using Microfluidic Chambers. Methods Mol. Biol. 1791, 157-168 (2018).
29. Tanimura, S. & Takeda, K. ERK signalling as a regulator of cell motility. J. Biochem. 162, 145-154 (2017).
30. Bornschlögl, T. How Filopodia Pull: What We Know About the Mechanics and Dynamics of Filopodia. Cytoskeleton 70, 590-603 (2013).
31. Syed, N. et al. Soluble neuregulin-1 has bifunctional, concentration-dependent effects on Schwann cell myelination. J. Neurosci. 30, 6122-6131 (2010).
32. Kidger, A. M. et al. Suppression of mutant Kirsten-RAS (KRASG12D)-driven pancreatic carcinogenesis by dual-specificity MAP kinase phosphatases 5 and 6. Oncogene 41, 2811-2823 (2022).
33. Leone, D. P. et al. Tamoxifen-inducible glia-specific Cre mice for somatic mutagenesis in oligodendrocytes and Schwann cells. Mol. Cell Neurosci. 22, 430-440 (2003).
34. Srinivas, S. et al. Cre reporter strains produced by targeted insertion of EYFP and ECFP into the ROSA26 locus. BMC Dev. Biol. 1, 4 (2001).
35. Feng, G. et al. Imaging neuronal subsets in transgenic mice expressing multiple spectral variants of GFP. Neuron 28, 41-51 (2000).
36. Bagnyukova, T. V. DUSP6 regulates drug sensitivity by modulating DNA damage response. Br. J. Cancer 109, 1063-1071 (2013).

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. A method for the treatment of central nervous system (CNS) injuries or CNS diseases that involve axonal disconnection, axonal lesion and/or axonal degeneration, comprising the step of administration of an activator of c-Jun N-terminal kinases (JNK) and/or of the transcription factor c-Jun to a subject to be treated, wherein the activator of JNK or c-Jun has the ability to convert mature oligodendrocytes (OLs) into repair OLs, thereby promoting axonal regrowth after injury of the CNS.

31. The method according to claim 30, wherein the activator of c-Jun has the ability to increase c-Jun expression, or to increase JNK activity in OLs.

32. The method according to claim 30, wherein the CNS injury or CNS disease is selected from the group consisting of spinal cord injury, optic nerve injury, traumatic brain injury, stroke, or neurodegenerative diseases of the CNS such as multiple sclerosis, amyotrophic lateral sclerosis, Parkinson's disease, glaucoma.

33. The method according to claim 32, wherein the CNS injury is spinal cord injury (SCI).

34. The method according to anyone of claim 30, wherein the activator of JNK and/or of c-Jun is a small molecule or a gene therapy agent.

35. The method according to claim 30, wherein the activator is mRNA.

36. The method according to claim 30, wherein the activator is mRNA comprising a nucleotide sequence that is at least partially identical to a nucleotide sequence of c-Jun tRNA, or active parts thereof.

37. The method according to claim 36, wherein the mRNA is complexed with lipid nanoparticles (LNPs), protein-based nanoparticles or virus-based nanoparticles.

38. The method according to claim 30, wherein the activator of c-Jun is an HDAC1/2 inhibitor, including, but not limited to Mocetinostat.

39. The method according to claim 30, wherein the activator of c-Jun is a regulator that mediates HDAC8 downregulation.

40. The method according to claim 30, wherein the activator of c-Jun is a Hif1alpha activator, including, but not limited to the prolyl hydroxylase inhibitor dimethyloxalylglycine.

41. The method according to claim 30, wherein the activator of JNK is a Hif1alpha activator, including, but not limited to the prolyl hydroxylase inhibitor dimethyloxalylglycine.

42. The method according to claim 30, wherein the activator of JNK is selected from the group consisting of Anisomycin, AEBSF hydrochloride, or Azaspiracid-1.

43. The method according to claim 30, wherein the activator of c-Jun or JNK is a Dual specificity protein phosphatase 6 or 6/1 (Dusp6 or Dusp6/Dusp1) inhibitor.

44. The method according to claim 43, wherein the Dusp6/Dusp1 inhibitor is BCI (C22H23NO).

45. The method according to claim 30, wherein the activator of c-Jun is a Dusp6-specific shRNA.

46. A pharmaceutical composition, comprising
a) an agent that activates JNK and/or upregulates the transcription factor c-Jun in OLs to convert OLs into repair OLs and thereby promote axonal regrowth after a CNS injury, or
b) repair OLs, and
c) a pharmaceutically acceptable carrier.

47. The pharmaceutical composition according to claim 46, wherein the composition comprises OLs and doxycycline in addition to the agent that activates JNK and/or upregulates the transcription factor c-Jun in OLs.

48. The pharmaceutical composition according to claim 46, wherein the activator of c-Jun or JNK is any one selected from
a) a small molecule or a gene therapy agent,
b) mRNA comprising a nucleotide sequence that is at least partially identical to a nucleotide sequence of c-Jun mRNA, or active parts thereof,
c) a HDAC1/2 inhibitor, including, but not limited to Mocetinostat,
d) a regulator that mediates HDAC8 downregulation,
e) a Hif1alpha activator, including, but not limited to the prolyl hydroxylase inhibitor dimethyloxalylglycine,
f) Anisomycin, AEBSF hydrochloride, or Azaspiracid-1,
g) Dusp6 or Dusp6/Dusp1 inhibitor, including, but not limited to BCI (C22H23NO).
